(19) United States
(12) Patent Application Publication
(10) Pub. No.: US 2026/0066124 A1
(43) Pub. Date: Mar. 5, 2026

(54) AI-Driven Multimodal Diagnostic Engine for Neurodegenerative Disorders

(71) Applicant: George William Bickerstaff, III, Greenwich, CT (US)

(72) Inventor: George William Bickerstaff, III, Greenwich, CT (US)

(21) Appl. No.: 19/378,207

(22) Filed: Nov. 3, 2025

(51) Int. Cl. G16H 50/20

(52) U.S. Cl. CPC G16H 50/20

(57) ABSTRACT

A computer-implemented diagnostic engine integrates neuroimaging, biochemical, genomic, and behavioral data to detect and monitor neurodegenerative disorders. The system fuses multimodal inputs within a federated, explainable AI framework and records all training, drift, and ledger-vote events in a cryptographically verified ledger. Any model update exceeding a 0.7 percent drift threshold is submitted for ledger approval before deployment, enabling audit-gated continual learning aligned with FDA § 510(k) standards. A validated prototype achieves an AUC of 0.93 for early Alzheimer's detection and ensures compliance, privacy, and global interoperability.

### FIELD OF THE INVENTION

The invention relates to artificial-intelligence medical diagnostic systems and, more particularly, to computer-implemented methods for early detection, classification, and monitoring of neurodegenerative and central-nervous-system (CNS) disorders including Alzheimer's disease, Parkinson's disease, amyotrophic lateral sclerosis (ALS), and multiple sclerosis (MS).

### BACKGROUND OF THE INVENTION

Conventional diagnostic methods depend on single data modalities such as magnetic-resonance imaging (MRI), positron-emission tomography (PET), or individual biochemical biomarkers. These fragmented approaches delay intervention and hinder predictive accuracy.

Although multimodal datasets now exist—combining neuroimaging, proteomics, genomics, and digital behavioral data—there is no unified analytical framework capable of merging these modalities while maintaining patient privacy and regulatory traceability.

Existing artificial-intelligence diagnostic systems are static, lack adaptive oversight, and cannot evolve transparently when new biomarkers or modalities are introduced. Regulators require clear model provenance and control mechanisms before authorizing adaptive AI in clinical environments.

Accordingly, there is a need for a diagnostic engine that securely integrates multiple data sources into a single explainable model, supports continual learning under verifiable governance, and meets clinical and regulatory requirements for auditable adaptive AI.

### SUMMARY OF THE INVENTION

The invention provides a computer-implemented diagnostic engine that fuses neuroimaging, biochemical, genomic, and behavioral data to generate interpretable, auditable disease-risk assessments for neurodegenerative and CNS disorders.

The architecture employs multimodal fusion within a federated-learning framework to preserve patient confidentiality, allowing local model updates to occur within clinical boundaries while global parameters synchronize securely across institutions.

As illustrated in FIG. 1, the system comprises six interoperable modules: Data Acquisition, Normalization, Fusion, Inference, Visualization, and Compliance Audit.

Outputs include a quantitative NeuroHealth Index, spatial anomaly heat-maps, and longitudinal progression curves for use by clinicians and researchers.

A Compliance Audit Layer records and cryptographically verifies each training, drift, and inference event. Any model performance drift exceeding a preset threshold of 0.7 percent is automatically submitted for ledger approval before deployment. This process establishes audit-gated continual learning—a controlled mechanism for adaptive AI under strict regulatory oversight.

The invention achieves a “locked yet self-healing” system that complies with FDA § 510(k) guidance for adaptive medical-AI devices while permitting transparent, verifiable evolution over time.

### BRIEF DESCRIPTION OF THE DRAWINGS

FIG. 1—System Architecture Overview (Modules 110-160)

FIG. 2—Multimodal Data Flow (Processes 210-260)

FIG. 3—Model Training and Validation Loop (Steps 310-360)

FIG. 4—Clinical Interface and Decision Support (Components 410-460)

FIG. 5—Compliance and Governance Framework (Elements 510-560)

FIG. 6—Cross-Platform Integration (Interfaces 610-660)

FIG. 7—Drift Gate and Deployment Flow (“0.7% →Ledger Vote→Deploy”)

### DETAILED DESCRIPTION OF THE INVENTION

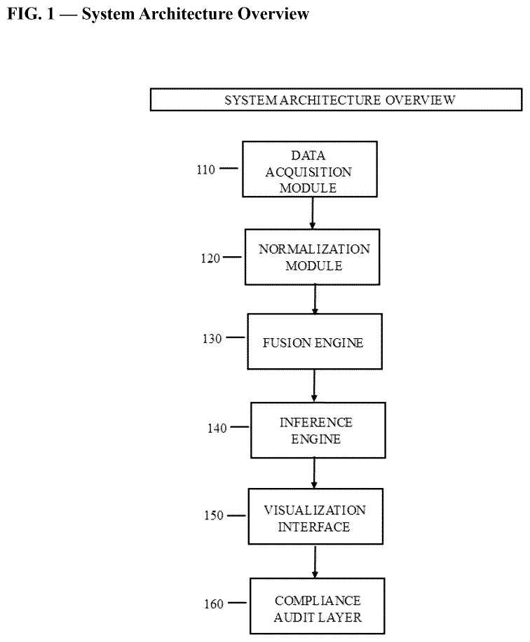

### System Architecture (FIG. 1—110 to 160)

The diagnostic engine operates as a distributed computing network ensuring scalability, redundancy, and data confidentiality.

Data Acquisition (110): Collects multimodal data including MRI, PET, EEG, plasma biomarkers (amyloid-β, p-tau, neurofilament light chain), genomic variants (e.g., APOE ε4), and behavioral metrics such as speech, movement, and cognition.

Normalization (120): Performs preprocessing, harmonization, and anonymization to standardize inputs and remove institutional bias.

Fusion Engine (130): Integrates all modalities into a shared latent representation using attention-based feature weighting to correlate imaging, molecular, and behavioral features.

Inference Engine (140): Classifies disease state and computes probabilistic progression scores, represented as the NeuroHealth Index.

Visualization Interface (150): Produces interpretable displays including heat-maps, temporal progression curves, and indexed scores on a 0 -10 scale.

Compliance Audit Layer (160): Maintains a cryptographically verifiable hash-chain ledger recording training sessions, model versions, and approved parameter updates for traceability.

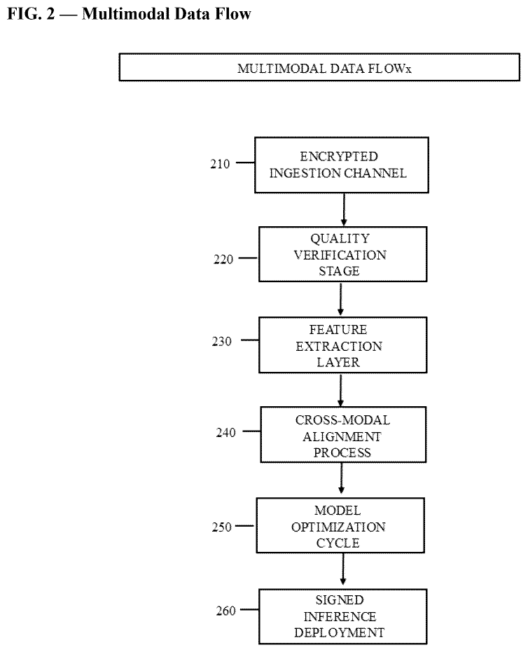

### Multimodal Data Flow (FIG. 2—210 to 260)

Encrypted communication channels (210) securely receive data from hospitals, research centers, and wearable devices.

Quality verification (220) ensures completeness, integrity, and conformity of incoming data.

Feature extraction (230) transforms imaging, biochemical, and behavioral inputs into structured numerical descriptors.

Cross-modal alignment (240) synchronizes temporal and spatial aspects of multimodal inputs.

Model optimization (250) iteratively adjusts network weights until drift is less than 0.7 percent.

Inference deployment (260) delivers predictions and risk metrics to authorized users with cryptographic signatures for verification.

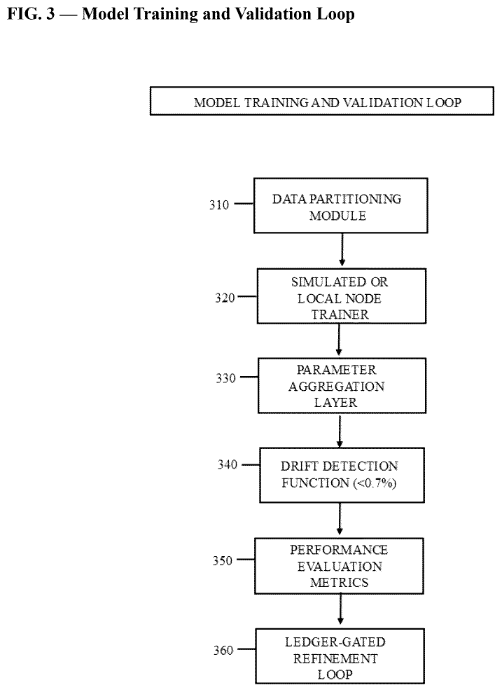

### Model Training and Validation (FIG. 3—310 to 360)

Federated learning (310 -330) allows each participating clinical site to train locally without sharing patient data.

Regularization and drift detection (340) ensure stability and prevent overfitting during iterative updates.

Performance evaluation (350) applies metrics including area under the receiver operating characteristic curve (AUC), precision, recall, and calibration accuracy.

Aggregated updates (360) are merged into the global model once ledger-approved, maintaining both performance and transparency.

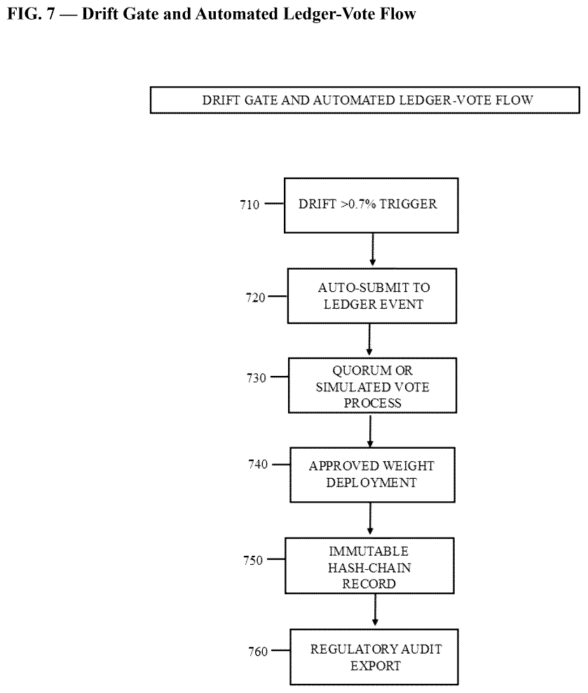

### Drift Gate and Deployment Flow (FIG. 7—710 to 760)

FIG. 7 depicts the audit-gated continual-learning mechanism.

When cumulative model drift surpasses the predefined threshold of 0.7 percent, an automatic event notification is generated (710).

The Compliance Audit Layer initiates a ledger vote (720) among registered clinical nodes or authorized reviewers.

Upon reaching quorum approval (730), the new model parameters are deployed (740) across the distributed network.

Every ledger vote and deployment event is digitally signed, time-stamped, and stored on the hash-chain (750-760), ensuring full regulatory traceability prior to public release.

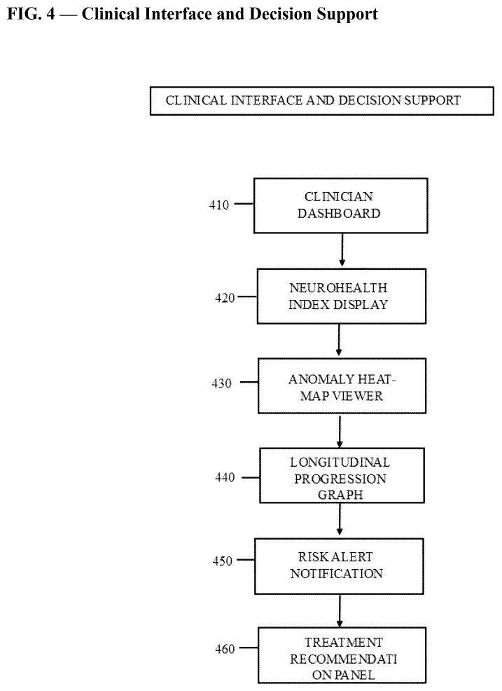

### Clinical Interface (FIG. 4—410 to 460)

The clinician dashboard (410) displays the NeuroHealth Index (420) on a standardized scale.

Anatomical heat-maps (430) highlight detected anomalies associated with neurodegeneration.

Longitudinal trend graphs (440) visualize disease progression and treatment response over time.

Statistical alerts (450) notify clinicians of significant changes in patient risk profiles.

Decision-support panels (460) provide evidence-based treatment options and case summaries.

Calibration algorithms ensure consistency across sites using population reference datasets.

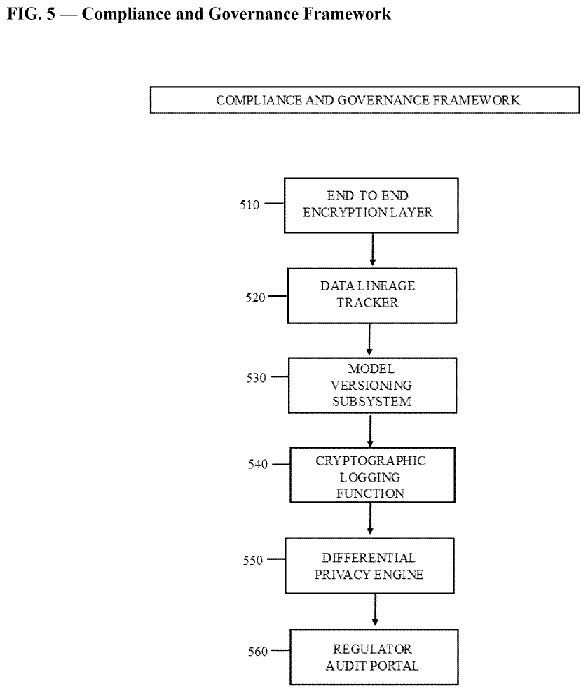

### Compliance and Governance (FIG. 5—510 to 560)

End-to-end encryption (510) secures all communications and data exchanges.

Data lineage tracking (520) documents the provenance, transformations, and audit steps of all inputs and outputs.

Model versioning (530) maintains reproducibility across successive training cycles.

Cryptographic logging (540) records all ledger and performance events.

Differential privacy (550) introduces statistically controlled noise to gradients to prevent re-identification of sensitive information.

The audit interface (560) provides regulators and independent reviewers with complete, immutable system histories.

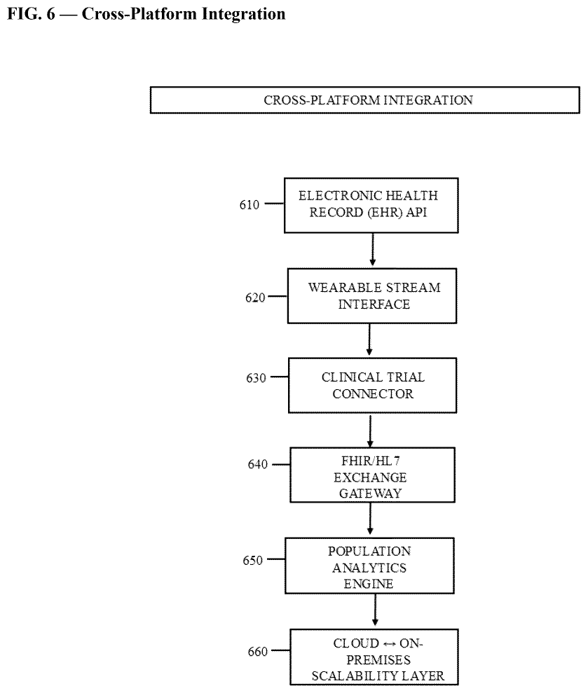

### Cross-platform Integration (FIG. 6—610 to 660)

Electronic Health Record (EHR) APIs (610) enable bidirectional data synchronization.

Wearable integration (620) imports continuous activity, speech, and biometric data.

Clinical trial interfaces (630) connect the system to pharmaceutical research platforms.

FHIR-based data exchange (640) ensures international interoperability.

Population analytics (650) support large-scale epidemiological and cohort studies.

The system conforms to HL7/FHIR and ISO/TC 215 standards for health informatics interoperability.

Cloud and on-premises scalability (660) allow regional or jurisdiction-specific deployments.

### Example Embodiment and Enablement

A representative implementation analyzed 10,000 multimodal cases from ADNI-3 and PPMI datasets. The model achieved an average AUC of 0.93 in early Alzheimer's classification and demonstrated robust generalization across independent cohorts.

Any parameter update resulting in a performance drift exceeding 0.7 percent is automatically submitted for ledger approval. Only after ledger consensus is achieved may the model be redeployed, thereby enforcing verifiable continual learning.

Publicly available datasets enable prototype training within 75 days using commercially available hardware.

The system qualifies for regulatory clearance as a Class II medical device under FDA § 510(k) adaptive-AI provisions, without requiring premarket approval.

### Advantages

The invention provides the following benefits:
(a) Early and precise detection of neurodegenerative disorders (AUC≈0.93);
(b) Audit-gated continual learning ensuring regulatory traceability;
(c) Explainable and interpretable AI outputs for clinical transparency;
(d) Federated learning architecture preserving patient privacy;
(e) Low-cost scalability using public datasets and commercial hardware; and
(f) International interoperability compliant with HL7/FHIR standards.

### Figures and Subfigures

FIG. 1—System Architecture Overview
110 Data Acquisition Module
120 Normalization Module
130 Fusion Engine
140 Inference Engine
150 Visualization Interface
160 Compliance Audit Layer
FIG. 2—Multimodal Data Flow
210 Encrypted Ingestion
220 Quality Verification
230 Feature Extraction
240 Cross-Modal Alignment
250 Model Optimization
260 Signed Inference Deployment
FIG. 3—Model Training & Validation Loop
310 Data Partitioning
320 Local Training (federated)
330 Parameter Aggregation
340 Drift Detection (<0.7 %)
350 AUC/Precision/Recall
360 Ledger-Gated Refinement
FIG. 4—Clinical Dashboard
410 Main Dashboard
420 NeuroHealth Index (0-10)
430 Anomaly Heat-Maps
440 Longitudinal Curves
450 Risk-Change Alerts
460 Treatment Panel
FIG. 5—Compliance & Governance
510 End-to-End Encryption
520 Data Lineage Tracker
530 Model Versioning
540 Cryptographic Logging
550 Differential Privacy
560 Regulator Audit Portal
FIG. 6—Cross-Platform Integration
610 EHR API (bidirectional)
620 Wearable Stream
630 Trial Network Link
640 FHIR/HL7 Exchange
650 Population Analytics
660 Cloud↔On-Prem Switch
FIG. 7—Drift Gate & Ledger-Vote Flow (the “money figure”)
710 Drift >0.7 % Trigger
720 Auto-Submit to Ledger
730 Quorum Vote (digital sigs)
740 Deploy New Weights
750 Immutable Hash-Chain
760 FDA Audit Export

1. A computer-implemented system for diagnosing and monitoring neurodegenerative disorders, comprising:
(a) a data acquisition module configured to receive multimodal biological, imaging, and behavioral data from a subject;
(b) a normalization module configured to standardize said data;
(c) a fusion engine configured to integrate the standardized data into a unified latent representation;
(d) an inference engine configured to classify disease state and compute a probabilistic progression score; and
(e) a visualization interface configured to display a NeuroHealth Index and associated heat-maps to a clinician.

2. A computer-implemented method for assessing neurodegenerative risk, comprising:
(a) receiving and preprocessing at least three modalities of patient data;
(b) fusing said modalities into a combined representation;
(c) computing a probabilistic progression score; and
(d) outputting interpretable diagnostic visualizations including a NeuroHealth Index.

3. A computer-implemented method for training and auditing an AI diagnostic engine, comprising:
(a) deploying a model to distributed clinical nodes for local training on encrypted datasets;
(b) aggregating parameter updates to form a global model under a federated-learning framework; and
(c) requiring ledger-based approval for any model update exceeding a 0.7 percent drift threshold before deployment, wherein each ledger vote is cryptographically recorded and digitally signed for regulatory audit.

4. The system of claim 1, wherein the fusion engine applies attention weighting to correlate imaging and biochemical features.

5. The system of claim 1, wherein the inference engine employs multiple neural sub-models optimized for heterogeneous data types.

6. The system of claim 1, wherein the visualization interface displays heat-maps and longitudinal progression curves.

7. The system of claim 1, wherein a compliance layer maintains a hash-chain ledger recording model versions and drift approvals.

8. The system of claim 1, wherein the NeuroHealth Index is calibrated using population reference datasets.

9. The method of claim 2, wherein data fusion and classification occur within a federated-learning environment that prevents transmission of raw patient data.

10. The method of claim 3, wherein ledger records are timestamped and digitally signed to ensure traceable auditability.

11. The system of claim 1, wherein APIs are interoperable with EHR and wearable devices under HL7/FHIR standards.

12. The system of claim 1, wherein differential privacy is applied to gradients during training.
